(19) United States
(12) Patent Application Publication
(10) Pub. No.: US 2007/0281001 A1
(43) Pub. Date: Dec. 6, 2007

(54) Cat in heat cool formula™

(76) Inventor: Herman Douglas, Norristown, PA (US)

Correspondence Address: Herman Douglas, 2nd Floor, 1121 South Divinity Street, Philadelphia, PA, 19143, US

(21) Appl. No.: 11/444,225

(22) Filed: May 31, 2006

(51) Int. Cl. A61K 33/00; A23K 1/165

(52) U.S. Cl. 424/442; 424/718

(57) ABSTRACT

An added vitamins that is found in PURINA CAT CHOW® and KNO3 to animal food products is to provided a natural and safe formula for cats and other animals having heat cycles, due to hormones changes that need cooling by a male or by surgical sterilization, known as spading. This composition formula is therefore a potent an important object of this invention to provide a safe and natural product to ease the emotional, sexual, physical and mental needs of the pet without surgical sterilization.

### REFERENCES CITED {REFERENCED BY}

References Cited {Referenced By}
U.S. Patent Documents

| | | |
|---|---|---|
| 3119691 | January 1964 | Ludington et al. |
| 3202514 | August 1965 | Burgess et al. |
| 3595666 | July 1971 | Dunning et al. |
| 3617300 | November 1971 | Borochoff et al |
| 3653908 | April 1972 | Buck et al |
| 3745023 | July 1973 | Greenbreg et al |
| 3857968 | December 1974 | Haas et al. |
| 3950543 | April 1976 | Buffa et al. |
| 4013775 | March 1977 | Nelson et al. |
| 4190679 | February 1980 | Coffee et al. |
| 4267195 | May 1981 | Boudreau et al. |
| 4282254 | August 1981 | Franzen et al. |
| 4391829 | July 1983 | Spradlin et al. |
| 4393085 | July 1983 | Spradlin et al. |
| 4418086 | November 1983 | Marino et al. |
| 4540585 | September 1985 | Priegnitz et al. |
| 4713250 | December 1987 | Tonyes et al. |
| 4810506 | March 1989 | Lewis et al. |
| 4857339 | August 1989 | Maselli et al. |
| 5631032 | May 1997 | Gil et al. |
| 5972668 | October 1999 | Georg et al. |
| 6,926,917 | Aug. 9, 2005 | Parthasarathy et al. |

Foreign Patent Documents

| | | |
|---|---|---|
| 4306514 | October | DE |
| 252730 | October | EP |
| 2111366 | October | GB |

### BACKGROUND

The primary ingredients are VITAMINS found in PURINA CAT CHOW® AND KNO3 added to the regular ingredients found in traditional dry cat food or other pet and human foods adding the right amount of ground PURINA CAT CHOW® to KNO3 this mixed formula would cool the animal sexual needs without male breeding. By adding to PURINA CAT CHOW® 1 pound ground dry food or semi moist mixed with 12 teaspoons of KNO3 produce a complete formula that will control the pet heat cycle. By giving the pet ½ teaspoon two time a day will remove the heat cyclefrom the pet.

There are many foods that are produced for pets. However there are no complete formula that can calm the pet when nature call for natural needs that the pet has for mating. When the pet need a male or female to reproduce there are no help for the pet through the pet food that are being presently manufactured. There are great efforts to get to the need for the cat to consume the food through taste and smell. This effort is called palatability increased.

The present vitamins are other nutritional ingredients have been found in general food for pets. This patent application formula product will cool the heat in the cat cycles. The vitamins and other nutrients and the right amount of KNO3 will cool the hormones that cause the cat to be in heat. It is therefore an important object of this invention to provide a safe and natural and comfortable product to control the sexual nature of the animal. This recipe or formula reduces the sexual urges in cats, dogs and human. These heat cycles are cause by hormones flair up, that causes the cat to be stimulated and irritated This emotional and negative behavioral effect the cat and the owner relationship. The formula is therefore a potent an important object of this invention to provide a safe and natural product to ease the emotional, sexual, physical and mental needs of the pet. This formula enable the pet owner to delay the surgical sterilization of the pet, often referred to as spading. Today there is no formula for cats that are developed to stop this problem of the heat cycle in cats. There is no alternative to surgical sterilization. This invention would be the first of it's kind to give the pet owner an alternative to the surgical sterilization. This surgical procedure is referred to as spaying or the use of the medical term ovariohysterectomy (OHE) This invention will give the cat owner some time to plan for the surgical sterilization. Although this invention can be use instead of spaying the pet. Cats owners in particular can extend the time for cats and other animal to be operated on. In this tight economic, the cost of spading have risen just as every thing has increased in cost. The waiting time and the actual operation will be prolong and the pet misery will also be passed on to the owners. It will be almost like the cat will be punishing the owner for not getting the pet spayed or getting the Douglas formula This formula is the first perfect formula ever developed. It revolutionized the cat care industry and will also help the owner to control the crisis that the owner have with the pet heat cycle. This formula will enable the cat to relax. This formula have many advantages and no disadvantages. There is no side effects. It's economically feasible and gives the pet owner more time to make preparation to get the cat spayed if the owner desire it. This unique formula are making many cats and animals lover to adore their pets more than ever before. Because the cat lover just found a product that really works and bring results. This invention gives the pet owner more control over their pet sexual drive that comes with crying, irritable and uncomfortable feelings. This recipe will cause less sexual pain in the cat and therefore less pain for all involved. This invention will help the pet to reduce sexual anxieties and the pet owner also. If the pet can have less pain the family gets more rest because when the animal is clam and out of sexual pain every one are happier. The cat can sleep and the pet owner can sleep everybody wins when the owner of the pet uses the relaxing formula.

This invention will helps to eliminate these kinds of pain complaints that cats owners gets when cat heat cycles began. “This cat is running me crazy, I must go to work in the morning and the cat is in heat and is crying and will continue to cry and is rubbing against everything in sight, and dragging her button on the floor, she will pace and be anxious all night long. She will continue this behavior until the operation be done. What will I do until the spading is done? Well give the cat this formula and the cat will be calm and restful and you can get the rest you so dearly desire. You can search the world and they will not be able to give you this calming formula for quieting your cat. This question floats around in many people heads that are looking for the answers to deal with cats that are in heat. HOW DO YOU SOOTHE A CAT IN HEAT? Use the Dr. H. Douglas “CAT IN HEAT COOL FORMULA”. The slogan” TAKE THE HEAT OUT OF THE CAT WITH THE PERFECT “CAT IN HEAT COOL FORMULA™”. The formula in essence are a sexual control solution as in female contraceptives that have been in use for many years. Already all over the world, the use of oral contraceptives has been developed into a business factor that cannot be ignored. Especially in view of the fact that the world population is continuing to grow upward, father development of the hitherto proven methods for birth control is absolutely necessary in the cat and other animals populations. This invention meets a need in the market place and therefore vast potential in reducing sexual pains in animals.

This invention should be found in any general food store. If your store do not carry it demand that they order it. The Veterinary services will be able to give you this formula until you can receive permanent surgical sterilization. Although a long as the cat consume the formula through its food, its oil ball, or directly through its mouth, and through the vitamin there will be no need for surgical sterilization. The cat will remain emotional and sexually calm and healthy. This invention can be used for other pets and human.

### TECHNICAL FIELD

The invention relates generally to oral supplement preparations, and more specifically to a low grams of vitamins and KNO3 removes sexual anxieties out of animals especially cats and even human.

This invention, an improved dry grounded pet food with added Ingredients comprises of said KNO3 to cat food Preferred embodiment comprises of balanced cat food such as PURINA CAT CHOW® added to KNO3 changes the ordinary cat food formula with elements that comfort the cat when in heat cycles. This invention has not been discovered by any one of the cat food manufactures or any one else for that matter according to the prior art. This present formula or recipe composes of 1 pound of PURINA CAT CHOW® and 12 teaspoons of KNO3 mixed with dry pet grounded or semi-moist cat food having substances to meet the physical, and emotional total and complete needs of the animals. Up to now, the dry pet food have only met the physical needs of the pet but not the regulatory sexual emotional needs of the pet. This invention meets the total needs of the pet. There are no claims in the prior art that claimed this adaptable invention for controlling the heat cycles.

The present invention adds to the concerns itself with the missing ingredients that are found in today pet foods. This improved dry cat food can meets the total palatability and emotional well being of the pet, and a process for the production of such food. In addition, this process is more economical and allows for higher throughput than all other prior art techniques The mixture of KNO3 convert ordinary pet food into an revolutionary cool formula by converting regular pet dry food into powdery form so when the cat consume the formula it can be quickly received into the pet natural digested system. This revolutionary invention may in the future replace the high cost in spading the animal in controlling the heat cycles. This invention improves the dry animal food and increase the health of the animals. The food product for the animal has been enhanced therefore making it food more complete. The added compound KNO3 to the prior art changes the ordinary to greater enhancements for said pets.

The chemical compound potassium nitrate is a naturally occurring mineral source of nitrogen. It is a nitrate with chemical formula KNO3. This compound along with other natural elements can be used to control heat cycle in pets. Either by giving the cat a low dose or low gram directly into the cat mouth or by placing low gram on any pet food. The pet may not eat the formula found directly in the pet food because the pet has some discriminating taste. The cat will only eat the formula if it become real hungry. If this happen feed the pet the formula directly by placing the formula into the pet mouth. You can use the liquid formula or through the pet food in getting the pet to consume the product. You can place the formula by spreading the formula over the pet food. If your cat is intelligent, it will not eat the part of dry cat food with the formula place on it. Sometime the pet will leave the formula part in the cat dish and eat around the formula. This of course will force the owner to use other forms to feed the cat the formula. The pet some time behavioral is, just like children when medication are involved. They resist taking the medication or supplements. The pet has the same kind of resistance that happens with children responses to taking supplements or medicine.

Pet foods in general have been known for years to meet basic needs of pets, but you don't just meet the basic needs, but meet all the needs of your pet by giving it the healthiest product available. To day, those that are skilled in the art are well known and aware in many processes and formulations for preparing such products. There are room for improving the formulations within the art. Historically, there are various techniques have been many attempts to improve the total palatability and health benefits of pet food with KNO3 it would add a most beneficial element to pet food. U.S. Pat. No. 3,857,968 discloses a process for improving the palatability of animals foods, such as dog foods, through the enzymatic treatment of a mixture of fat and protein. The patent does not identify the reason for the improvement in palatability. It is disclosed, however, that the reaction produces a complex array of reaction products, which are responsible for the improvement. In none of the prior art are mentioned anything remotely close to the new improved pet dry food, semi-moist, and canned food that are found in this particular invention. It's thought that various sugar may improve the palatability of pet foods for reference U.S. Pat. No. 3,617,300 to Borochoffet al indicates that dextrose may improve the palatability of pet foods. According to the disclosed process, some of the starch content of a solid pet food is enzymatically converted to glucose directly in the dog food by alpha-amylase and amyloglucosidase. This patent teaches that a combination of these two enzymes permits the reaction to occur without adversely affecting the solid character of the dog food.

To date, their have not been any prior art to address the pet needs for its emotional well being.

In the past there have been developments to get the taste right so the animals can eat the food but no one have attempted to get the other needs of the pet. All have been focused on ingredients to increase the palatability of pets foods. No one focused on the emotional and sexual regularization of the other needs of the pets. It's true that many features contribute to the taste of pet food, it is also recognized that the texture or relative softeners of a pet food is also important. Softness can be a particular problem with dry pet foods. The prior art just leave the pet with a gap in it food intake. All the prior art just about do the same thing when it come to the well being of the pet. This invention will solve the problem of the total needs of the pet. The pet will be more relax and healthier and that is the goal of the present invention. The pets owner have left the emotional needs up to the veterinarian and have not cross into that line, but this can be very expense for the pet owner. By offering the pet owner this formula, even the vet will benefit. The vet can in all instant spread his time by using this formula and let his patient owners know there are other ways in treating the pet.

It is not necessary to follow the order of getting the pet to consume the formula, it can be administered in several ways. The preferred application is to place it into the formulation of the pet food. However this will call for a complex formulation of the formula. So the highest and simple form is to place the cat food in powder form into the pet mouth. It can be spread over regular pet food in small amounts as one would do salt and pepper or any other seasons. It can also be place on a small amount of oil ball, it can be place on ground up pet food and spread over the dry pet food. The pet owner must be aware that the cat have keen smell and keen taste and once the pet detect the formula in it dish it may not consume the food unless it is very hungry and the pet will not eat very much at one time. This invention is relatively flexible in application in administering this formula to the pet. Other sequences of getting the pet to consume the formula and using these methods will be obvious and may be followed.

A wide variety of suitable pet food will be suitable and are available for use in this invention. The combination of cat, dog and people food can be used in practicing this invention. The different foods are in the prior art but the new and novel invention by adding KNO3 it to the pet diet actually revolutionize the entire pet food industry. This sexual emotional formula completely relax the animals. This powder like material that are formulated from natural elements gives the animals the soothing feeling of comfort and no sexual needs what so ever. There are no side effects and it has only positive results upon the pets and other animals. This invention can also be used upon human Depending upon its concentration the KNO3 used in the present invention cost less than a dollar per teaspoon per finished product. This is thought to be more economical than some prior art techniques. The method of feeding the animal the KNO3 mixed formula will inactivate the animal sexual behavioral. The pet can be given the KNO3 with out mixing it with the PURINA CAT SHOW® but it is preferred to mixed it with other foods that provides complete balanced food for the pet.

Thus, there has been shown and desired various embodiments of the formula to enable the pet to consume the invention in accordance with the teachings of the present invention. The most unique and preferred method is taken orally in powered form, it can be consume through paste, liquid and solid mixture. There are Many changes, modifications and variations of the present invention and will, however, become apparent to those skilled in the art after considering this specification. All such changes, modifications, and variations that do not depart from the spirit and scope of the present invention are deemed to be the invention, which is limited only by the claims that follow:

I claim:

1. A multiphase combination food PURINA CAT CHOW®, composition consists of vitamins supplements mixed with said ingredients: omega fatty acids, vitamins, and antioxidants, protein, fat and carbohydrate and added to said elements KNO3 to the above ingredients to change for improvements for animals, comprising three phases, said three phases consisting of controlling heat cycles in animals dosages of KNO3 in cat vitamins and other healthy nutrients that animals needs, especially a cat in heat or other animal or human by feeding a cat, dog and or human in heat by feeding it human food or direct consuming it food with ½ teaspoon of said mixture formula as needed, by placing direct formula into the mouth with water following the feeding of the formula with the present of KNO3 formula through food, vitamins and or water or grease ball it formation in powered form, paste, liquid, and solid mixture. This compound is known as potassium nitrite and have been use in food products and other uses for years. However this compound has not been used with precise focused method as use in this invention. The KNO3 mixed with said PURINA CAT CHOW® forms a complete composition natural compounds are found to have positive effect upon the nervous system of animals as well as human being with the right amount that vary from pet to pets. A pinch of the formula have amazing effect upon the animal. The cat in heat are given KNO3 formula immediately upon observing the cat heat cycle beginning. The results are within hours of consumption of the formula. There is no known invention that get search calm and amazing results in animals.

2. The multiphase combination of claim 1 for ½ teaspoon of the said mixture show changes in the animal producing the calm in the animal in heat and produce the total effect of cooling the pet sexual heat cycle in minutes by administering the pet with the product KNO3 mixed with PURINA CAT CHOW®.

3. The said mixture comprises of multiphase combination claim 2 wherein the formula is feed in grounded dry cat food about ½ teaspoon full as the pet needs the formula to clam it. The said composition comprises of said KNO3 mixed with PURINA CAT CHOW® or any suitable pet food as long as the said ingredients embodied the complete elements that comprises this formula.
